(19) United States
(12) Patent Application Publication
(10) Pub. No.: US 2022/0175961 A1
(43) Pub. Date: Jun. 9, 2022

(54) IMPROVED THERAPEUTIC METHOD FOR RARE OCULAR DISEASES BY GENE REPLACEMENT

(71) Applicants: UNIVERSITE PARIS-SACLAY, Gif-sur-Yvette (FR); CENTRE NATIONAL DE LA RECHERCHE SCIENTIFIQUE, Paris (FR); RETINA FRANCE, Colomiers Cedex (FR); VARIANT, Paris (FR)

(72) Inventors: Jérôme ROGER, Orsay cedex (FR); Elodie Kim GRELLIER, Orsay cedex (FR)

(73) Assignees: UNIVERSITE PARIS-SACLAY, Gif-sur-Yvette (FR); CENTRE NATIONAL DE LA RECHERCHE SCIENTIFIQUE, Paris (FR); RETINA FRANCE, Colomiers Cedex (FR); VARIANT, Paris (FR)

(21) Appl. No.: 17/437,138

(22) PCT Filed: Mar. 9, 2020
(86) PCT No.: PCT/EP2020/056199

§ 371 Date: Sep. 8, 2021

(30) Foreign Application Priority Data
Mar. 8, 2019 (EP) 19305276.8
Oct. 24, 2019 (EP) 19306381.5

(51) Int. Cl. A61K 48/00; C07K 14/47; C12N 15/86

(52) U.S. Cl. CPC A61K 48/005; C07K 2319/41; C12N 15/86; C07K 14/4705

(57) ABSTRACT

A recombinant adeno-associated virus (AAV) vector that carries a nucleic acid sequence encoding the retinal transcription factor cone-rod homeobox (CRX) for its use in treating CRX-associated IRDs in a subject in need thereof or for use in treating inherited retinal dystrophies caused by hypomorphic mutations in the CRX target genes.

### FIELD OF INVENTION

The present invention relates to methods for treating retinal diseases and pharmaceutical compositions comprising CRX as an active agent.

### BACKGROUND OF INVENTION

Progressive loss of photoreceptors (PR) in inherited retinal diseases (IRDs) are common causes of visual impairment with a prevalence of 1/4,000, representing about 17,000 patients in France. They form a group of genetically and clinically heterogeneous diseases with more than sixty human genes identified. The efficacy and safety of gene therapies, by the mean of intraocular injections of Adeno-Associated Virus (AAV), have been proven with preclinical success translated into clinical effectiveness. Such approaches are mostly used in patients with recessive loss of function mutations. The goal is to compensate the mutated gene that causes the disease with a fully functional copy.

Dominant forms of PR degeneration represent at least 20% of retinitis pigmentosa (RP) cases. Nowadays, very limited number of therapeutic approaches have been initiated for these dominant forms. Indeed, dominant mutations may increase the activity or confer a new activity of the gene product. Therefore, in contrast with recessive forms, dominant forms of PR degeneration are more challenging to cure since restoring a missing function may not be sufficient. Dominant forms may indeed require expressing the healthy copy with the concomitant silencing of the mutated gene product.

More than fifty mutations in the human CRX gene are associated with autosomal dominant Leber congenital amaurosis (LCA), cone-rod dystrophy (CORD) and RP.

The transcription factor Cone-Rod Homeobox (CRX) plays a central role in regulating gene expression of rod and cone photoreceptors, the primary light sensing cells of the retina. Cone-rod homeobox (CRX) protein is a “paired-like” homeodomain transcription factor that is essential for regulating rod and cone photoreceptor transcription. CRX acts as a transcriptional activator by interacting with co-activators, promoting histone acetylation at target gene promoters and mediating enhancer/promoter intrachromosomal looping interactions of target photoreceptor gene.

In this context, it was surprisingly found that expression of the transcription factor CRX can be used for treating a subject carrying a CRX-associated disease, i.e. an inherited dystrophy associated with a dominant retinal mutation in CRX or subject carrying at least one hypomorphic mutation in at least one CRX target gene, without any side effect.

The present invention relates generally to recombinant viral vectors and methods of using recombinant viral vectors to express the CRX protein in the retina of subjects suffering from retinal diseases.

### SUMMARY OF THE INVENTION

This invention thus relates to a vector recombinant adeno-associated virus (AAV) vector comprising a nucleic acid sequence encoding the retinal transcription factor cone-rod homeobox (CRX) for its use in treating CRX-associated IRDs in a subject in need thereof.

In one embodiment of the invention, the AAV vector is an AAV2 serotype.

In a more specific aspect of the invention, the AAV vector is an AAV2/5 or AAV2/8 serotype.

In a further aspect, the polynucleotide comprised in the AAV vector of the invention is under the control of a promoter that drive expression in rod and cone photoreceptors chosen among Rhodopsin (Rho), beta-phosphodiesterase (PDE), retinitis pigmentosa 1 (RP1) or the human Rhodopsin kinase 1 (GRK1 or RK1) promoters.

In a more specific aspect, the polynucleotide is under the control of the GRK1 promoter.

In one embodiment, the vector of the invention is for use in treating CRX-associated IRDs wherein the CRX-associated IRD is chosen among a retinopathy resulting from a dominant mutation in the CRX gene or results in symptoms due to a hypomorphic mutation cured by the expression of CRX.

In a still further embodiment, the vector of the invention is used for treating a CRX associated disease wherein the CRX-associated IRD is chosen among retinitis pigmentosa, Leber's congenital amaurosis or cone-rod dystrophies or to hypomorphic mutations in CRX target genes such as PDE6B, NMNAT1, ARL13b, AIPL1 or ABCA4 genes.

In a further as aspect, the vector for use of the invention is administered to the subject in a therapeutically effective amount.

In one embodiment, the vector for use of the invention is administrated in quantity comprised is between 10⁸ and 10¹² vg/eye, more preferably between 1×10⁹ and 1×10¹² vg/eye.

In one embodiment, the vector of the invention is administered before disease onset.

In another embodiment, the vector of the invention is administered after initiation of photoreceptor degeneration.

In still further embodiment, the vector of the invention is administered as long as there are functional cone and/or rod photoreceptors.

In one embodiment, the vector for use of the invention is included in a composition, more particularly a pharmaceutical composition.

### Definitions

In the present invention, the following terms have the following meanings:
“AAV” is an abbreviation for adeno-associated virus, and may be used to refer to the virus itself or derivatives thereof.
“AAV-CRX or AAV-hRK1-CRX” refers to an AAV2/5 vector comprising the human Rhodopsin kinase 1 promotor driving the expression of the human CRX.
“AAV-GFP or AAV-hRK1-GFP” refers to refers to and AAV2/5 vectors comprising the human Rhodopsin kinase 1 promotor driving the expression of the GFP.
“CRX”, refers to cone-rod homeobox CRX (NCBI: Nucleotide NM_000554.6; Protein NP_000545.1, or Ensembl No. ENSG00000105392) is an Otd/OTX-like ‘paired’ homeodomain transcription factor that is preferentially expressed in vertebrate rod and cone photoreceptor cells in the retina and pinealocytes in the brain. CRX plays an essential role in the development and maintenance of functional mammalian rod and cone photoreceptors. CRX encodes for a 299 aminoacid protein that contains a homeodomain (HD) near its N-terminus that is responsible for DNA bonding. One of skill in the art would understand that a CRX coding sequence may include any nucleic acid sequence that encodes an CRX gene product. The CRX coding sequence may or may not include intervening regulatory elements (e.g.: introns, enhancers, or other non-coding sequences).
“Dominant inherited retinal diseases” refers to a mode of inheritance of a disease through families Dominant means that only one allele carrying the mutated genes is sufficient to trigger the disease examples of which are certain forms of RP, CORD and LCA.
“hCRX” refers to the human CRX gene.
“Heterologous gene” or “heterologous nucleotide sequence” will typically refer to a gene or nucleotide sequence that is not naturally-occurring in the virus. Alternatively, a heterologous gene or nucleotide sequence may refer to a viral sequence that is placed into a non-naturally occurring environment (e.g.: by association with a promoter with which it is not naturally associated in the virus).
“Hypomorphic mutation” refers to a mutation that confers a reduced level of activity to the altered gene product or that leads to a reduced level of expression of the altered gene product. Non-limitative examples of such mutations are certain subtype of Phosphodiesterase 6B (PDE6B) mutations, as well as others identified for instance in NMNAT1, ARL13b, AIPL1 or ABCA4 genes.
“ITR” or “inverted terminal repeat” refer to the stretch of nucleic acid sequences that exist in AAV vector and/or recombinant Adeno-Associated Viral Vectors (rAAV) that can form a T-shaped palindromic structure, that is required for completing AAV lytic and latent life cycles (Muzyczka N and Berns KI 2001). The term “non-resolvable ITR” refers to a modified ITR such that the resolution by the Rep protein is reduced. A non-resolvable ITR can be an ITR sequence without the terminal resolution site (TRS) which leads to low or no resolution of the non-resolvable ITR and would yield 90-95% of self-complementary AAV vectors (McCarty et al 2003).
“Operably linked” refers to a functional relationship between two or more polynucleotide (e.g., DNA) segments. Typically, the term refers to the functional relationship of a transcriptional regulatory sequence to a sequence to be transcribed. For example, a promoter or enhancer sequence is operably linked to a coding sequence if it stimulates or modulates the transcription of the coding sequence in an appropriate host cell or other expression system. Generally, promoter transcriptional regulatory sequences that are operably linked to a transcribed sequence are contiguous to the transcribed sequence, i.e., they are cis-acting. However, some transcriptional regulatory sequences, such as enhancers, need not be physically contiguous or located in close proximity to the coding sequences whose transcription they enhance
“Polynucleotide of interest” refers any nucleotide sequence coding for CRX.
“Promoter” refers to a sequence that regulates transcription of an operably-linked gene, or nucleotide sequence encoding a protein, etc. Promoters provide the sequence sufficient to direct transcription, as well as, the recognition sites for RNA polymerase and other transcription factors required for efficient transcription and can direct cell specific expression. In addition to the sequence sufficient to direct transcription, a promoter sequence of the invention can also include sequences of other regulatory elements that are involved in modulating transcription (e.g.: enhancers, kozak sequences and introns). In addition, standard techniques are known in the art for creating functional promoters by mixing and matching known regulatory elements. “Truncated promoters” may also be generated from promoter fragments or by mix and matching fragments of known regulatory elements.
“Subject” or “subject in need thereof” is refers to a mammal, preferably a human. In one embodiment, a subject may be a “patient”, i.e., a warm-blooded animal, more preferably a human, who/which is awaiting the receipt of, or is receiving medical care or was/is/will be the object of a medical procedure, or is monitored for the development of a disease. In one embodiment, the subject is an adult (for example a subject above the age of 18). In another embodiment, the subject is a child (for example a subject below the age of 18). In one embodiment, the subject is a male. In another embodiment, the subject is a female.
“Treating” or “treatment” of inherited retinal dystrophy refers (IRD), to ameliorating IRD such as by slowing or arresting or reducing its development or at least one of the clinical symptoms thereof. “Treating” or “treatment” can also refer to alleviating or ameliorating at least one physical parameter including those which may not be discernible by the patient. “Treating” or “treatment” can also refer to modulating the IRD, either physically, (e.g., stabilization of a discernible symptom), physiologically, (e.g., stabilization of a physical parameter), or both. More specifically, “treatment” of IRD means any action that results in the improvement or preservation of visual function and/or regional anatomy in a subject having IRD. “Treating” also encompasses “preventing or “prevention” as used herein, refers to preventing or delaying the onset or development or progression of the IRD. “Prevention” as it relates to IRD means any action that prevents or slows a worsening in visual function, retinal anatomy, and/or an IRD parameter, as described below, in a patient with IRD and at risk for said worsening. Methods for assessing treatment and/or prevention of disease are known in the art and described herein below.
“Virus vector” or “viral vector” is intended to refer to a non-wild-type recombinant viral particle that functions as a gene delivery vehicle and which comprises a recombinant viral genome packaged within a viral (e.g.: AAV) capsid. A specific type of virus vector may be a “recombinant adeno-associated virus vector”, or “AAV vector”.

The recombinant viral genome packaged in the viral vector is also referred to herein as the “vector genome”.

### DETAILED DESCRIPTION OF THE INVENTION

The present invention is based on the discovery on viral vectors that express a heterologous CRX gene in both cone and rod photoreceptor cells.

Accordingly, the present invention relates to a recombinant adeno-associated virus (AAV) vector that directs expression of the retinal transcription factor cone-rod homeobox (CRX) in cone and rod cells, compositions comprising such a vector and method of use of this vector or compositions.

The viral vector of the invention may be a recombinant adeno-associated (rAAV or AAV) vector. AAV vectors are small, single-stranded DNA viruses which require helper virus to facilitate efficient replication. The viral vector comprises a vector genome and a protein capsid. The viral vector capsid may be supplied from any of the AAV serotypes known in the art, including presently identified human and non-human AAV serotypes and AAV serotypes yet to be identified. Virus capsids may be mixed and matched with other vector components to form a hybrid viral vector, for example the ITRs and capsid of the viral vector may come from different AAV serotypes. In one aspect, the ITRs can be from an AAV2 serotype while the capsid is from, for example, an AAV2 or AAV5 serotype. In addition, one of skill in the art would recognize that the vector capsid may also be a mosaic capsid (e.g.: a capsid composed of a mixture of capsid proteins from different serotypes), or even a chimeric capsid (e.g.: a capsid protein containing a foreign or unrelated protein sequence for generating markers and/or altering tissue tropism).

It is contemplated that the viral vector of the invention may comprise an AAV2 capsid. It is further contemplated that the invention may comprise an AAV5 capsid.

According to the invention the term “AAV” refers to AAV type 1 (AAV-1), AAV type 2 (AAV-2), AAV type 3 (AAV-3), AAV type 4 (AAV-4), AAV type 5 (AAV-5), AAV type 6 (AAV-6), AAV type 7 (AAV-7), and AAV type 8 (AAV-8) and AAV type 9 (AAV9). The genomic sequences of various serotypes of AAV, as well as the sequences of the native terminal repeats (TRs), Rep proteins, and capsid subunits are known in the art. Such sequences may be found in the literature or in public databases such as GenBank. See, e.g., GenBank Accession Numbers NC_001401 (AAV-2), AF043303 (AAV2), and NC_006152 (AAV-5).

As used herein, an “AAV vector” refers to an AAV vector comprising the polynucleotide of interest (i.e. a heterologous polynucleotide) for the genetic expression in a cone and/or rod cell. The rAAV vectors contain 5′ and 3′ adeno-associated virus inverted terminal repeats (ITRs), and the polynucleotide of interest (CRX) operatively linked to sequences, which regulate its expression in a target cell.

In a specific aspect, the AAV vector of the invention is an AAV2 serotype.

In another aspect, the AAV vector of the invention is an AAV2/5 or AAV2/8 serotype.

The AAV2/5 or AAV2/8 vectors of the invention are produced using methods known in the art. In short, the methods generally involve (a) the introduction of the AAV vector into a host cell, (b) the introduction of an AAV helper construct into the host cell, wherein the helper construct comprises the viral functions missing from the AAV vector and (c) introducing a helper virus into the host cell. All functions for AAV virion replication and packaging need to be present, to achieve replication and packaging of the AAV vector into AAV virions. The introduction into the host cell can be carried out using standard virological techniques simultaneously or sequentially. Finally, the host cells are cultured to produce AAV virions and are purified using standard techniques such as cesium chloride (CsCl) gradients. Residual helper virus activity can be inactivated using known methods, such as for example heat inactivation. The purified AAV virion is then ready for use in the methods of the invention.

The vector may also comprise regulatory sequences allowing expression and, secretion of the encoded protein, such as e.g., a promoter, enhancer, polyadenylation signal, internal ribosome entry sites (IRES), sequences encoding protein transduction domains (PTD), and the like. In this regard, the vector comprises a promoter region, operably linked to the polynucleotide of interest, to cause or improve expression of the protein in infected cells. Such a promoter may be ubiquitous, tissue-specific, strong, weak, regulated, chimeric, inducible, etc., to allow efficient and suitable production of the protein in the infected tissue. The promoter may be homologous to the encoded protein, or heterologous, including cellular, viral, fungal, plant or synthetic promoters.

The preferred promoter for use in the present invention is chosen among promoter able to drive expression in cone and rod cells such as retinitis pigmentosa 1 (RP1) or the human Rhodopsin kinase 1 (GRK1).

In a preferred aspect the invention deals with an AAV vector wherein the polynucleotide encoding CRX is under the control of the human Rhodopsin kinase 1 (GRK1) promoter.

An exemplary amino acid sequence for CRX is NCBI Reference Sequence: NP_000545.1 and an exemplary nucleic sequence for CRX is NCBI Reference Sequence: NM_000554.6.

The invention also deals with compositions and pharmaceutical compositions comprising the viral vectors of the invention. The pharmaceutical composition are formulated together with a pharmaceutically acceptable carrier. The compositions can additionally contain one or more other therapeutic agents that are suitable for treating or preventing, for example, CRX-associated IRD. Pharmaceutically acceptable carriers enhance or stabilize the composition, or can be used to facilitate preparation of the composition. Pharmaceutically acceptable excipient refers to a non-toxic solid, semi-solid or liquid filler, diluent, encapsulating material or formulation auxiliary of any type. Pharmaceutically acceptable excipients that may be used in the compositions of the invention include, but are not limited to solvents, surfactants, dispersion media, coatings, antibacterial and antifungal agents, isotonic and absorption delaying agents, and the like that are physiologically compatible ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, silica, colloidal silica, magnesium trisilicate, polyvinyl pyrrolidone, cellulose-based substances (e.g.: sodium carboxymethylcellulose), polyethylene glycol, polyacrylates, waxes, polyethylene-polyoxypropylene-block polymers, polyethylene glycol and wool fat.

The pharmaceutical composition according to the present invention may further comprise antioxidant agents, including, but not limited to, ascorbic acid, ascorbyl palmitate, butylated hydroxytoluene, potassium sorbate or Rosmarinus officinalis extracts.

The pharmaceutical composition according to the present invention may further comprise pharmaceutically acceptable salts, including, but not limited to, acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The excipient can also be a solvent or dispersion medium containing, for example, water, ethanol, polyol (e.g.: glycerol, propylene glycol, and liquid polyethylene glycol, and the like), suitable mixtures thereof, and vegetables oils such as oleic acid. The proper fluidity can be maintained, for example, by the use of a coating, such as lecithin (i.e., soy lecithin or de-greased soy lecithin), by the maintenance of the required particle size in the case of dispersion and by the use of surfactants.

The vector or the pharmaceutical composition of the present invention can be administered by a variety of methods known in the art. The route and/or mode of administration vary depending upon the desired results. It is preferred that administration be subretinal. The pharmaceutically acceptable carrier should be suitable for subretinal, intravitreal, intravenous, sub-cutaneous or topical administration.

The composition should be sterile and fluid. Proper fluidity can be maintained, for example, by use of coating such as lecithin, by maintenance of required particle size in the case of dispersion and by use of surfactants. In many cases, it is preferable to include isotonic agents, for example, sugars, polyalcohols such as mannitol or sorbitol, and sodium chloride in the composition.

Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives may be included, as required. For delayed release, the vector may be included in a pharmaceutical composition, which is formulated for slow release, such as in microcapsules formed from biocompatible polymers or in liposomal carrier systems according to methods known in the art.

Pharmaceutical compositions of the invention can be prepared in accordance with methods well known and routinely practiced in the art.

Pharmaceutical compositions are preferably manufactured under GMP conditions. Typically, a therapeutically effective dose or efficacious dose of the viral vector is employed in the pharmaceutical compositions of the invention. The viral vectors may be formulated into pharmaceutically acceptable dosage forms by conventional methods known to those of skill in the art. Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

Actual dosage levels of the active ingredients in the pharmaceutical compositions of the present invention can be varied so as to obtain an amount of the active ingredient which is effective to achieve the desired therapeutic response for a particular patient, composition, and mode of administration, without being toxic to the patient. The selected dosage level depends upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors.

The present invention provides a method for treating a CRX-associated IRD in a subject in need thereof, by delivering into the eye of the subject an AAV vector comprising a polynucleotide encoding CRX which when expressed in cone and rod cells, has a beneficial effect on the retinal disease.

More particularly, the present invention provides a method for treating a CRX-associated IRD in a subject in need thereof, wherein CRX-associated IRD relates either to a retinopathy resulting from a dominant mutation in the CRX gene or results in symptoms due to a hypomorphic mutation cured by the expression of CRX, and even more particularly for treating dominant forms of retinal degenerative diseases such as RP, CORD or LCA or for treating a hypomorphic mutation in PDE6B.

According to a further aspect, the invention deals with an AAV vector expressing CRX, for use in treating a CRX-associated IRD in a subject in need thereof.

According to the present invention, CRX-associated IRD relates either to a retinopathy resulting from a dominant mutation in the CRX gene or results in symptoms due to a hypomorphic mutation cured by the expression of CRX.

Typically, the subject is affected or likely to be affected with a CRX-associated IRD resulting from a dominant mutation in the CRX gene. Dominant mutations in this gene are associated with dominant forms of retinal degenerative diseases such as RP, CORD or LCA.

According to another aspect, the invention also deals with a vector or a composition or a pharmaceutical composition for use in treating CRX-associated IRDs, wherein said IRD results from a dominant mutation in the CRX gene.

According to another aspect, the invention also deals with a vector or a composition or a pharmaceutical composition for use in treating CRX-associated IRDs, wherein said IRD is caused by a hypomorphic mutation in a CRX target gene.

In a more specific aspect, the invention also deals with a vector or a composition or a pharmaceutical composition for use in treating a hypomorphic mutation in PDE6B.

According to the present invention the treatment is either a therapeutic treatment or prophylactic or preventative treatment.

Accordingly, the subject treated are either those already with the IRD as well as those prone to have the disease or those in whom the disease is to be prevented.

In one embodiment, a subject is successfully “treated” if, after receiving a therapeutically effective amount of a compound according to the present invention, the subject shows observable and/or measurable reduction one or more the symptoms associated with the specific disease; and/or improvement in quality of life.

The parameters for assessing successful treatment and improvement in the disease are readily measurable by routine procedures familiar to a physician.

According to another aspect, the vector, the composition or the pharmaceutical composition of the invention is administrated in a therapeutically effective amount.

A physician can start doses of the viral vectors of the invention employed in the pharmaceutical composition at levels lower than that required to achieve the desired therapeutic effect and gradually increase the dosage until the desired effect is achieved. In general, effective doses of the compositions of the present invention, for the treatment of CRX-associated retinal dystrophy as described herein vary depending upon many different factors, including means of administration, target site, physiological state of the patient, whether the patient is human or an animal, other medications administered, and whether treatment is prophylactic or therapeutic.

As used herein, “therapeutically effective amount” means an amount of AAV vector that provides a therapeutic benefit to the subject. In one embodiment, the term “effective amount” means level or amount of vector that is aimed at, without causing significant negative or adverse side effects to the target, (1) delaying or preventing the onset of CRX-associated IRDs; (2) slowing down or stopping the progression, aggravation, or deterioration of one or more symptoms of CRX-associated IRDs (3) bringing about ameliorations of the symptoms of CRX-associated IRDs; (4) reducing the severity or incidence of CRX-associated IRDs; or (5) curing the CRX-associated IRDs.

The doses of vectors may be adapted depending on the disease condition, the subject (for example, according to his weight, metabolism, etc.), the treatment schedule, etc.

A preferred effective dose within the context of this invention is a dose allowing an optimal expression in PR cells.

Treatment dosages need to be titrated to optimize safety and efficacy. For subretinal administration with a viral vector, the dosage may range from 1×10⁸ vector genomes (vg)/eye to 1×10¹² vg/eye. For example the dosage may be, 1×10⁸ vg/eye, 1, 5×10⁸ vg/eye, 2×10⁸ vg/eye, 2.5×10⁸ vg/eye, 3×10⁸ vg/eye, 4×10⁸ vg/eye, 5×10⁸ vg/eye, 6×10⁸ vg/eye, 7.5×10⁸ g/eye, 8×10⁸ vg/eye, 9×10⁸ vg/eye, 0.5×10⁹ vg/eye, 1×10⁹ vg/eye, 1.5×10⁹ vg/eye, 2.5×10⁹ vg/eye, 3×10⁹ vg/eye, 4×10⁹ vg/eye, 5×10⁹ vg/eye, 6×10⁹ vg/eye, 7×10⁹ vg/eye, 7.5×10⁹ vg/eye, 8×10⁹ vg/eye, 9×10⁹ vg/eye, 0.5×10¹⁰ vg/eye, 1×10¹⁰ vg/eye, 1.5×10¹⁰ vg/eye, 2.5×10¹⁰ vg/eye, 3×10¹⁰ vg/eye, 4×10¹⁰ vg/eye, 5×10¹⁰ vg/eye, 6×10¹⁰ vg/eye, 7×10¹⁰ vg/eye, 7.5×10¹⁰ vg/eye, 8×10¹⁰ vg/eye, 9×10¹⁰ vg/eye, 0.5×10¹¹ vg/eye, 1×10¹¹ vg/eye, 1.5×10¹¹ vg/eye, 2.5×10¹¹ vg/eye, 3×10¹¹ vg/eye, 4×10¹¹ vg/eye, 5×10¹¹ vg/eye, 6×10¹¹ vg/eye, 7×10¹¹ vg/eye, 7.5×10¹¹ vg/eye, 8×10¹¹ vg/eye, 9×10¹¹ vg/eye, 0.5×10¹² vg/eye, 1×10¹² vg/eye.

According to a preferred embodiment of the invention, the vectors or the composition comprising said vector is administrated in a quantity between 1×10⁹ and 1×10¹² vg/eye.

Administering the vector of the invention vector to the subject may be done by direct subretinal injection for an expression of CRX in cone and rod cells.

According to another aspect, the vector or the composition for use according to the invention is administered before disease onset and as long as there is a need to prevent this onset.

According to another aspect, the vector or the composition for use according to the invention is administered after initiation of photoreceptor degeneration more particularly after initiation of photoreceptor degeneration as long as there are functional cone and/or photoreceptors and as long as there is a need.

The viral vectors described herein are mainly used as one-time doses per eye, with the possibility of repeat dosing to treat regions of the retina that are not covered in the previous dosing. The dosage of administration may vary depending on whether the treatment is prophylactic or therapeutic.

The various features and embodiments of the present invention, referred to in individual sections and embodiments above apply, as appropriate, to other sections and embodiments, mutatis mutandis. Consequently, features specified in one section or embodiment may be combined with features specified in other sections or embodiments, as appropriate.

The invention will be further illustrated by the following figures and examples. However, these examples and figures should not be interpreted in any way as limiting the scope of the present invention.

### BRIEF DESCRIPTION OF THE DRAWINGS

FIG. 1: 822-pKL.AAV.GRK1.hCRX final vector plasmid map comprising the human Rhodopsin kinase 1 promoter driving the expression of human CRX. The promoter was subcloned using the restriction sites SpeI and EcoRI. The cDNA coding for human CRX was subcloned using KpnI digestion generating a cohesive site, which was blunted, and follow-up to XbaI digestion.

FIG. 2: The selected AAV vector combined with the selected promoter allows efficient transduction of WT photoreceptors and Crx^{Rip/+} immature cone-like photoreceptors.

WT or Crx^{Rip/+} mice received a single subretinal injection of 2.5×10¹⁰ AV-GFP at P30. Eyes were harvested 14 days after for sectioning and immunolabelled with an anti-GFP antibody. Nuclei were stained with DAPI.

ONL: Outer Nuclear Layer; INL: Inner Nuclear Layer; GCL: Ganglion Cell Layer. Scale bar, 20 μm.

FIG. 3: Overexpression of CRX following subretinal injection of AAV-CRX in Crx^{Rip/+}.

Crx^{Rip/+} mice received or not a single subretinal injection of three different doses of AAV-CRX (0.5×10¹⁰, 1×10¹⁰ and 2.5×10¹⁰ vg per eye) at P30. CRX expression was assessed 14 days after by immunoblotting.

FIG. 4: Quantification of CRX expression following subretinal injection of AAV-GFP or AAV-CRX.

Crx^{Rip/+} mice received a single subretinal injection of three different doses of AAV-GFP or AAV-CRX at 0.5×10¹⁰, 1×10¹⁰ or 2.5×10¹⁰ vg per eye between P30 and P40. CRX expression was quantified 14 days after by immunoblotting. Expression was normalized using Tubulin. All quantifications were reported to endogenous CRX expression in animals injected with 0.5·10¹⁰ vg of AAV-GFP.

Black bars: endogenous CRX expression in Crx^{Rip/+} injected AAV-GFP; white bars: CRX expression in Crx^{Rip/+} mice injected AAV-CRX.

FIG. 5: Differentiated PR can be observed in Crx^{Rip/+} retina using AAV-CRX vector.

Crx^{Rip/+} mice received a single subretinal injection of 2.5×10¹⁰ AAV-CRX at P30. The other eye was not injected and used as control. Eyes were harvested 1 months after for sectioning and labelled with anti-Rhodopsin and anti-cone arrestin antibodies. Nuclei were stained with DAPI.

ONL, outer nuclear layer; INL, inner nuclear layer; GCL, ganglion cell layer Scale bar, 20 μm.

FIG. 6: Differentiated PR with outer segment can be observed in Crx^{Rip/+} retina using a lower dose of AAV-CRX vector (0.5·10¹⁹ vg).

(A) Both eyes of Crx^{Rip/+} mice received a subretinal injection of 0.5×10¹⁰ vg of either AAV-CRX or AAV-GFP at P30. Eyes were harvested 2 months after and flat mounted after labelling with anti-Rhodopsin and anti-cone arrestin antibodies. GFP expression following AAV-GFP subretinal injection was directly observed. Scale bar, 20 μm. (B) Crx^{Rip/+} mice received a single subretinal injection of 0.5×10¹⁰ AAV-CRX at P30, as well as a small dose (<1·10⁹ vg) of AAV-GFP to sparsely labelled transduced photoreceptors with GFP and visualize their morphology. Confocal acquisition of multiple z sections allowed 3D reconstruction.

1, outer segment; 2, nucleus; 3, synaptic terminal Scale bar, 5 μm.

FIG. 7: AAV-CRX subretinal injection allowed restoration of photoreceptor light sensitivity.

Crx^{Rip/+} mice received or a single subretinal injection of 0.5×10¹⁰ AAV-CRX or AAV-GFP at P30 in both eyes. Light response was assessed 4 months after injection. (A) The increase of scotopic b-wave response was statistically significant in AAV-CRX treated animals compared to AAV-GFP controls at the stimulus intensity of 2.2 log cd·sec/m². (B) The increase of photopic b-wave response was statistically significant in AAV-CRX treated animals compared to AAV-GFP controls at the stimulus intensity of 1.6 log cd·sec/m². (C) The light/dark choice test showed that the number of time than Crx^{Rip/+} mice injected with AAV-CRX crossed the line toward the light compartment is comparable to non-injected WT mice. In contrast, Crx^{Rip/+} mice injected with AAV-GFP crossed the line more often indicating a default in their ability to detect the light.

Grey bar, non-injected WT; black bars, Crx^{Rip/+} mice injected AAV-GFP; white bars, Crx^{Rip/+} mice injected AAV-CRX *p<0.05, **p<0.01.

FIG. 8: Photoreceptor degeneration was reduced in rd10 mice following AAV-CRX vector injection at the dose of 2.5·10¹⁰ vg.

(A) AAV-CRX vector was injected or not in P14 Rd10 mice. Retinas were collected two months after injection. Retinal sections were immunolabelled with anti-Rhodopsin (Rho) and anti-cone arrestin antibodies (CA). Nuclei were stained with DAPI.

(B) Histograms represent the measurement of rd10 ONL thickness injected with AAV-CRX (white bar) or not (NI, black bar).

ONL, outer nuclear layer; INL, inner nuclear layer; GCL, ganglion cell layer. Scale bar, 20 μm.

FIG. 9: Photoreceptor degeneration was reduced in rd10 mice following AAV-CRX vector injection at the dose of 0.5·10¹⁰ and 1·10¹⁰ vg.

Histograms represent the measurement of rd10 ONL thickness at 2 months after injection at P14 with AAV-CRX. The specific detection of human CRX on paraffin sections allowed the discrimination of a transduced or non-transduced area.

Black bars, AAV-CRX non-transduced area; white bars, AAV-CRX transduced area.

FIG. 10: Expression of CRX^{R41W} in a cone-only retina led to cone degeneration which can be prevented using AAV-CRX.

(A) Histogram representing the decreased photopic b-wave amplitude in 2-months old Nrl^{-/-}; Tg(cRX^{R41W}) compared to Nrl^{-/-} at the light stimulus of 2.8 log cd·sec/m². (B) Histogram representing the preservation two months post-injection of the photopic b-wave amplitude in Nrl^{-/-}; Tg(CRX^{R41W}) treated at 2-months with AAV-CRX compared to AAV-GFP. *p<0.05.

### EXAMPLES

The present invention is further illustrated by the following examples.

### Example 1

Material and Methods Animals

Male and female adult (3-8 weeks old) C57BL/6J, Crx^{Rip/+} and Tg(CRX^{R41W}) mices, and male and female young (2 weeks old) rd10 mices were produced in the laboratory animal facilities (Animalerie central-campus CNRS Gif sur Yvette).

All animal experiments were carried out according to European guidelines for the care and use of experimental animals, and were approved by the regional ethics committee (CEEA59).

### Vectors

rAAV2/5 vectors expressing green fluorescent protein (GFP) or human cone-rod homeobox protein (CRX) under the control of the human rhodopsin kinase (GRK1) promotor were produced.

1. Vector Plasmid Cloning (822-pKL.AAV.GRK1.hCRX Vector #6556 Batch)

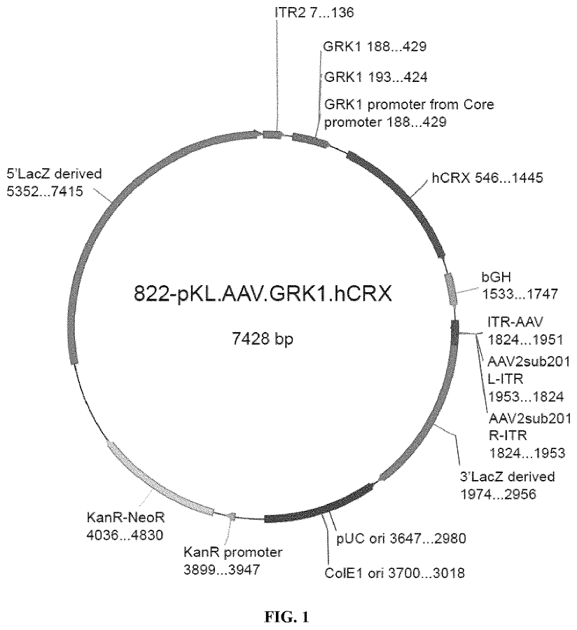

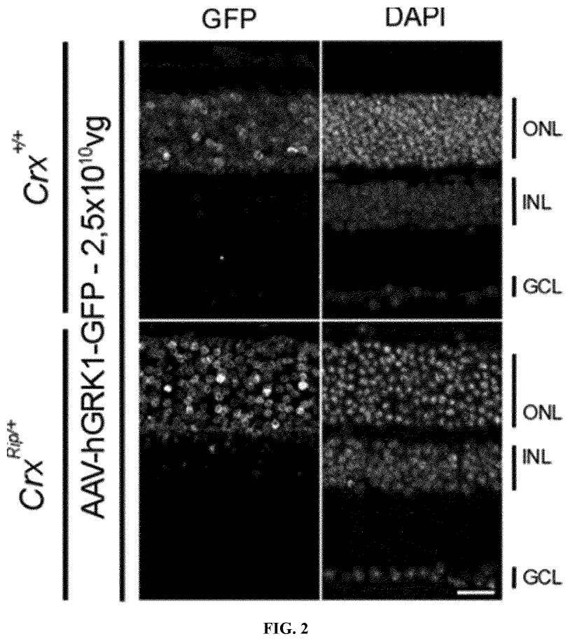

The vector plasmid was constructed in 2 phases as described below:
First, the promoter GRK1 was extracted from the 754-pAAV.GRK1.eGFP.wpre.bGH by double digestion SpeI (position 185) and EcoR1 (position 427) and inserted in the 778-pKL.AAV.CAG.bGH vector plasmid in advance double digested by SpeI (position 188)-EcoR1 (position 1947) to extract the CAG promoter and allow to the intermediate plasmid pKL.AAV.bGH. The GRK1 promoter was inserted in the pKL.AAV.bGH generating the 821-pKLAAV.GRK1.bGH backbone plasmid.
Second step: the 821-pKLAAV.GRK1.bGH backbone plasmid was linearized by double digestion EcoR1 (position 531)/XbaI (position 569). The hCRX gene was extracted from the pcDNA4c.hCRX plasmid by KpnI digestion (position 1171), generating a cohesive site, which was blunted, and follow-up to XbaI digestion (position 2120). The extracted hCRX gene was inserted in the linearized pKL.AAV.hCRX.bGH at position 531 to generate the 822-pKL.AAV.GRK1.hCRX final vector plasmid (FIG. 1).
The 822-pKL.AAV.GRK1.hCRX final vector plasmid has been sequencing from position 106 to 2745 bp corresponding to the expression cassette.

### 2. AAV2/5 Manufacturing and Purification Operating Procedure

### Cell Amplification, Transfection, Harvest and Supernatant PEG-Precipitation

The HEK293 cells seeding in CellStack Cells 5 chambers with vent caps (CS5) are cultured with DMEM supplemented with 10% FBS and 1% Pen/Strep. At approx. 80% of confluency, cells are co-transfected with the vector plasmid and helper plasmid (containing helper genes from adenovirus (E2A, E4 and VA RNA) and the rep cap genes corresponding to the serotype-2 for rep and capsid serotype-5) using the CaPO₄ precipitate technique. The culture medium is removed from the CS5 and exchanged with the transfection medium; the cells are then incubated 6 to 15 hours at 37+/−1° C. and 5+/−1% CO₂. The transfection medium is then removed from the CS5 and replaced by fresh exchange medium (DMEM, 1% Pen/Strep) prior to a 3 days incubation at 37+/−1° C. and 5+/−1% CO₂. The cells of the CS5 transfected are then harvested. The supernatant is only precipitated at 5+/−3° C. overnight with PEG. The precipitated supernatant is then centrifuged. The supernatant is discarded and the PEG-pellet is resuspended in TBS before benzonase digestion.

### Vector Purification by Double CsCl-Gradient Ultracentrifugation

The viral suspension is centrifuged and the vector-containing supernatant is loaded on a step density CsCl gradient in Ultra-Clear tube for SW28 rotor. The gradient is centrifuged at 28 000 rpm for 24 hours at 15° C. The full particles band is collected and transferred to a new Ultra-Clear tube for SW41 rotor. The 2^{nd} gradient is centrifuged at 38 000 rpm for 48 hours. The enriched-full particles band is then collected. The viral suspension is then subjected to 4 successive rounds of dialysis in a Slide-a Lyzer cassette against buffer solution for ophthalmic preparation. The purified vector is finally collected, sampled for vg titer and purity assay, and stored at <−70° C. in polypropylene low-binding cryovials.

### 3. Quality Control: Vector Genome Quantification

The basic unit to determine the vector dose is vector genome (vg). The vector genome corresponds to the concentration of the particles containing the gene of interest and is quantified by quantitative PCR using ITR-2 specific primers, described in D'Costa et al., 2016 paper.

### In Vivo Sub-Retinal AAV Injections

Adult/young mice were anesthetized with intraperitoneal injection, using 50 μL/10 μg per mice of a solution composed of xylazine (1 mg/mL) and ketamine (10 mg/mL) in phosphate-buffered saline (PBS). Iris were dilated using specific eye drops (Mydriaticum (Tropicamide 5 mg 0.5%) and Neosynephrine (Phenylephrine hydrochloride 2.5%)). Eyes were anesthetizing using Cebesine 0.4%.

Once anesthetized, C57BL/6J, rd10 and Crx^{Rip/+} mice were injected into the subretinal space with:
AAV2/5.hGRK1.GFP (AAV-GFP) at:
   5×10⁹ vg per eye (n=20 Crx^{Rip/+} mice; n=3 rd10 mice; n=2 Nrl^{-/-} Tg(CRX^{R41W})
   1×10¹⁰ vg per eye (n=8 Crx^{Rip/+} mice, n=2 rd10 mice)
   2.5×10¹⁰ vg per eye (n=3 C57BL/6J mice; n=10 Crx^{Rip/+} mice; n=2 rd10 mice) or
AAV2/5.hGRK1.hCRX (AAV-CRX) at:
   b 5×10⁹ vg per eye (n=3 C57BL/6J mice; n=21 Crx^{Rip/+} mice; n=3 rd10 mice; n=2 Nrl^{-/-)}
   1×10¹⁰ vg per eye (n=9 Crx^{Rip/+} mice; n=4 rd10 mice)
   2.5×10¹⁰ vg per eye (n=6 C57BL/6J mice; n=10 Crx^{Rip/+} mice; n=14 rd10 mice)

A 33 G beveled needle attached to a 5 μL Hamilton syringe was inserted into the ventral subretinal space passing through the RPE/choroid membrane, near to the optic nerve. 2 μL were slowly injected to detach the retina from RPE cells (during approximately 30 seconds).

### Protein Extraction and Immunoblot Analysis

Mice were euthanized using cervical dislocation. Retinas were dissected and frozen in dry ice, and then stored at −80° C. Proteins were extracted using a lysis buffer (20 mM Na₂HPO₄, 250 mM NaCl, 5% DTT, 30 mM NaPPi, 0.1% NP-40, 5 mM EDTA) with protease inhibitors (cOmplete™). Mechanical disruption was achieved by sonication and proteins were isolated after centrifugation (10 minutes, 13200 rpm, 4° C.). 20 μg of protein from each eye studied were loaded on a 7.5%-12% gradient acrylamide gel (Biorad). The run was carried out at constant voltage (100 V). Upon completion, western blot transfer was carried out using an iBlot 2 Dry Blotting System on nitrocellulose membrane (ThermoFisher Scientific).

After transfer, the nitrocellulose membrane was incubated with blocking buffer (PB ST [PBS, 0.05% Triton X-100], 5% milk) for 1 hour at room temperature. Primary antibody was then added in fresh blocking buffer, either overnight at 4° C. (mouse anti-Crx (A-9), ref.: sc-377138 [Santa Cruz Biotechnology, Inc.], batches J1116 and H2918, dilution 1/5000) or for 1 hour at room temperature (mouse anti-tubulin clone DM1A, ref.: T9026 [Sigma-Aldrich], batch 052M4837, dilution 1/10000). Excess of primary antibody was rinsed 3 times with PBST for 10 minutes.

Secondary antibody was added in fresh blocking buffer for 2 hours at room temperature (goat anti-mouse IgG-peroxidase, ref.: A4416 [Sigma-Aldrich], batch SLBH3692, dilution 1/5000), and then rinsed 3 times for 10 minutes with PBST.

ECL revelation kit (Supersignal™ West Dura Extended Duration Substrate: 34076 [ThermoFisher Scientific]; batch SK256986) was added on the nitrocellulose membrane 5 minutes before revelation in dark room (Carestream® Kodak® BioMax® light film, ref.: Z373508 [Sigma-Aldrich]).

Membrane can be stripped with stripping buffer (1.5 g glycine, 1 mL SDS 10%, 1 mL Tween-20, milliQ water qs. 100 mL, pH 2.2). As such, the membrane was incubated twice for 10 minutes at room temperature, followed by two PBS washes for 10 minutes, and finally washed twice with PBST for 5 minutes. Membranes were incubated with primary and secondary antibodies as previously described. After revelation, films were scanned and analyzed with Image J.

### Histological Analysis

Mice were euthanized using cervical dislocation. Eyes were collected and fixed in 4% paraformaldehyde (PFA) for 1 hour, rinsed in PBS before dehydratation and paraffin inclusion. 7 μm-thick sections were cut with a microtome (Rotatory microtom, ref.: HM 340E [ThermoFisher Scientific]), placed overnight at 37° C., and then stored at room temperature. Prior to immunohistochemistry (IHC), slides were deparaffinized and then incubated in hot citrate buffer (pH 6, 0.1 M) for 20 minutes at 500 W. Once cooled down, slides were placed in PBS. For immunostaining (IHC) on flat mount retina, the retina was dissected and fixed in 4% PFA for 1 hour, rinsed in PBS and incubated directly with the primary antibody.

Primary antibodies in Dako REAL™ Antibody diluent (ref 52022 lot 20060091) supplemented with 0.3% Triton X-100 were added to the slides overnight at 4° C. in a dry chamber (mouse anti-rhodopsin clone 4D2, ref.: MABN15 [Merck], batch 2935495, dilution 1/1000; rabbit anti-cone arrestin, ref.: AB15282 [Merck], batch 2802590, dilution 1/1000; goat anti-GFP, ref.: ab6673 [Abcam], dilution 1/1000). Flat mount retinas, were incubated with the primary antibodies for 2 days.

They were rinsed 3 times with PBST, 5 minutes before adding secondary antibodies (donkey anti-mouse IgG Alexa Fluor 555, ref.: A31570 [ThermoFisher Scientific], dilution 1/1000; donkey anti-rabbit IgG Alexa Fluor 488, ref.: A21206 [ThermoFisher Scientific], dilution 1/1000; donkey anti-goat Alexa Fluor 488, ref.: A11055 [ThermoFisher Scientific], dilution 1/1000) in Dako REAL™ Antibody diluent (ref S2022 lot 20060091) supplemented with 0.3% Triton X-100. After 2 hours of incubation at room temperature in the dark, slides were rinsed 3 times with PBST for 5 minutes, and DAPI (ref.: 62248 [ThermoFisher Scientific], batch RJ2279362, dilution 1/1000) was added for 20 minutes in PBST. Finally, slides were rinsed 3 times with PBST. Flat mount retinas were incubated for 1 night with the secondary antibody.

For flat mount retina, the retina was positioned on the slide with photoreceptor side up. FluorSave™ Reagent (ref.: 345789, Millipore, batch 3034632) was used to add a coverslip. Slides were stored at 4° C. Pictures were acquired using an Imager M2 microscope (Zeiss) or a LSM710 confocal microscope (Zeiss) and analyzed using Zen software and Image J.

### Electroretinogram

Electroretinogram (ERG) recordings were performed using a focal ERG module attached to Micron IV (Phoenix Research Laboratory). Briefly, mice were dark-adapted overnight and prepared for the experiment under dim-red light. The mice were anesthetized with ketamine (100 mg/kg) and xylazine (10 mg/kg) and received topical proparacaine hydrochloride (0.5%, Alcon) via eye drops. Pupils were dilated with tropicamide (1%, Alcon) and phenylephrine (2.5%, Alcon) and lightly coated with GONAK hypromellose ophthalmic demulcent solution (2.5%, Akorn). Lens of the Micron IV was placed directly on the cornea, and a reference electrode was placed on the mouse head. Scotopic responses were elicited with a series of flashes of increasing light intensities from −1.7 to 2.2 cd·s/m2. Photopic responses were elicited under rod-desensitizing background light with a series of flashes of increasing light intensities from −0.5 to 2.8 cd·s/m2. Values of a- and b-wave were extracted and plotted for comparisons between groups of interest.

### Light-Dark Choice.

The apparatus had 20 cm-high Plexiglas walls and consisted of a brightly lit white box (40×15 cm) connected by a trap door (6×6 cm) to a dark box (15×15 cm). Illumination in the dark box was <10 Lux. In the light box, the light was placed at the end of the 40 cm compartment, thus providing a gradient of illumination increasing from 600 Lux at the entrance to 1500 Lux at end of compartment.

Testing was performed between 9 am and 12:00. Each mouse was placed in the dark compartment for 10s, the trap door was then opened and mice allowed to freely explore the whole apparatus for 5 min. Step through latency, number of entries and total time spent in the light compartment were scored.

### Example 2

Selection of the promoter and the serotypes of the AAV vector to be used to efficiently transduced photoreceptors in CRX-associated retinopathy mouse models.

Several AAV serotypes and promoters have been tested and showed transduction of mature photoreceptors in adults. In the case of Crx^{Rip/+} retina, the outer nuclear layer contains only immature cone-like photoreceptors and therefore AAV vectors commonly used to treat differentiated photoreceptors may not be as efficient. Therefore, we tested several serotypes expressing GFP driven by a photoreceptor-specific promoter, which directs gene expression in both rod and cone photoreceptors. The human Rhodopsin kinase 1 (GRK1) promoter was selected because i) it is already well characterized to express genes in both rod and cone photoreceptors ii) it is already used in clinical trial (PDE6B) iii) It is active in our mouse models based on our published transcriptomic data. For the serotype, we selected the AAV2/5 vector described as transducing specifically mouse photoreceptors.

Different doses of GFP-expressing AAV vectors, ranging from 1×10⁹ to 5×10¹⁰ vg per Crx^{Rip/+} eye, were injected into the subretinal space at post-natal day 30 (P30). The quality of the subretinal injection was confirmed immediately after by the presence of a retinal detachment observed by Optical Coherence Tomography (OCT). Two weeks later, retinal detachment disappeared and the efficacy of photoreceptor transduction was assessed using fluorescent fundus imaging followed by histological analysis. A GFP immunofluorescence analysis performed on sections from injected retina revealed a strong GFP expression in the injected area restricted to the outer nuclear layer were photoreceptors are located (FIG. 2).

In conclusion, the AAV vector serotype selected for future therapy is an AAV2/5 vector including a GRK1 promoter.

### Example 3

Quantification of CRX expression driven by GRK1 promoter using an AAV2/5 vector.

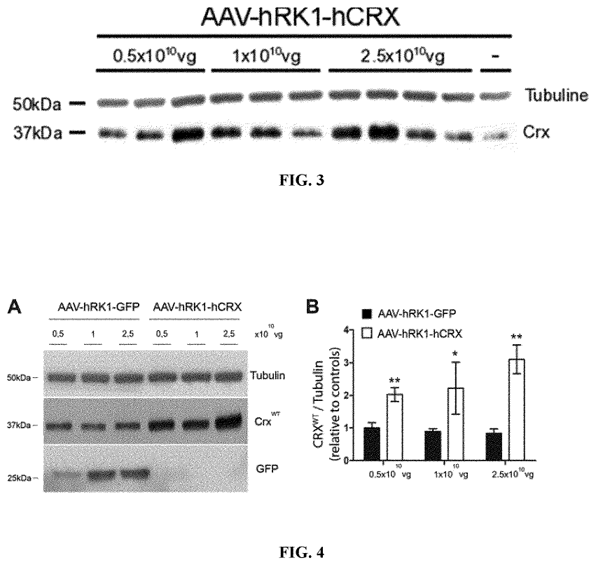

In order to verify that the product (CRX-expressing AAV vector or AAV-CRX) is able to produce CRX protein, a series of subretinal injections was performed in Crx^{Rip/+} retina with three different doses (0.5·10¹⁰, 1·10¹⁰ and 2.5·10¹⁰ vg) and CRX expression was assessed 14 days later. At all three tested doses, increased expression of CRX was observed. The amount produced was more dependent of the injection than the dose used (FIG. 3). This experiment was repeated when the subretinal injection technic was fully mastered but this time GFP-expressing vector (AAV-GFP) was injected as control (FIG. 4) at three different doses (0.5·10¹⁰, 1·10¹⁰ and 2.5·10¹⁰ vg). CRX relative expression was quantified after tubulin normalization and reported to endogenous CRX expression following AAV-GFP injection at 0.5·10¹⁰ vg. CRX expression was clearly significantly increased after AAV-CRX injection proportionally to the dose used.

### Example 4

Restoration of photoreceptor differentiation in Crx^{Rip/+} retina following AAV-CRX injection.

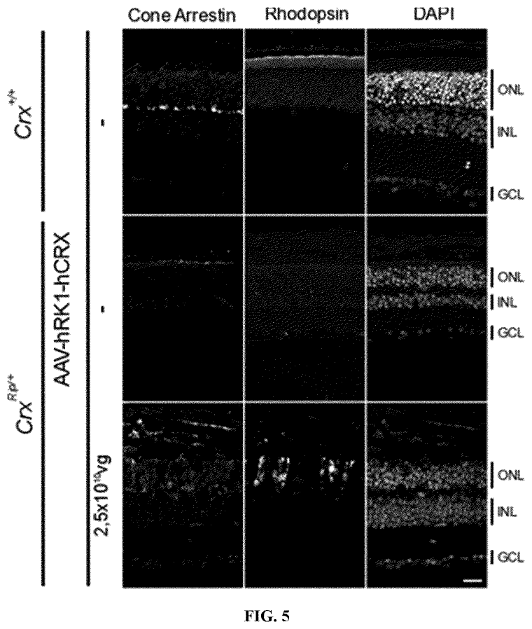

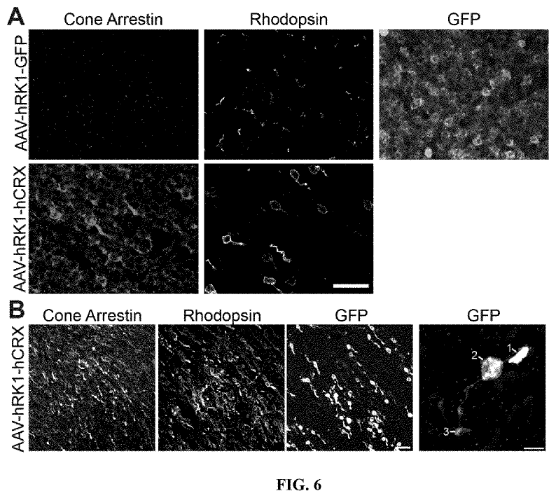

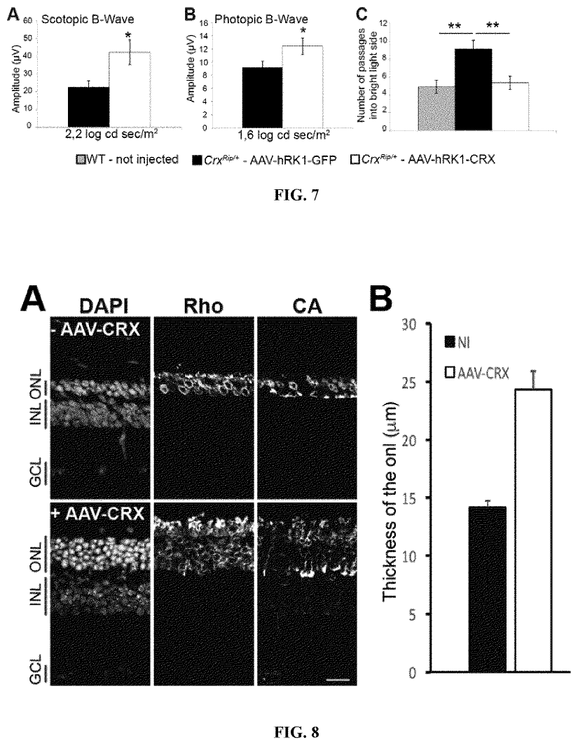

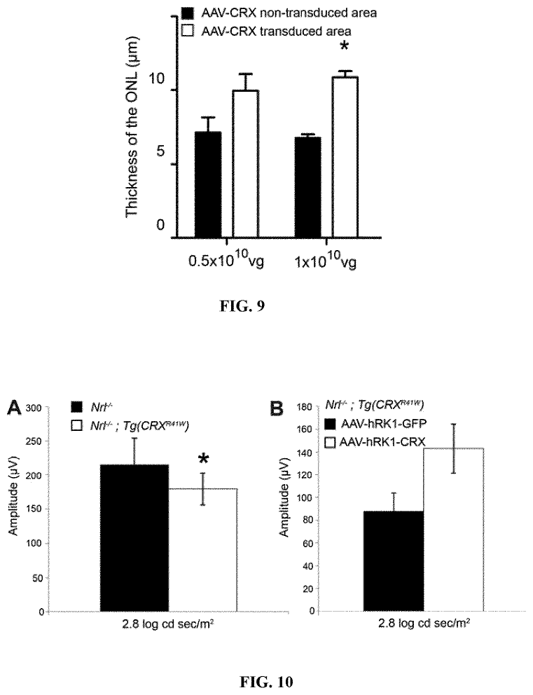

To assess the ability of the product (CRX-expressing AAV vector or AAV-CRX) to restore photoreceptor differentiation, AAV-CRX was injected at 2.5×10¹⁰ vg per eye at P30. Retinas were collected two months after for histological analysis Immunohistochemistry analysis was performed using anti-rhodopsin and anti-cone arrestin to label rod and cone photoreceptors, respectively. The result clearly revealed a large area of Rhodopsin- and Cone Arrestin-positive cells around the injected area (FIG. 5). Imaging of Rhodopsin- and Cone-Arrestin-positive cells on flat mount retina four months after AAV-CRX subretinal injection in Crx^{Rip/+} demonstrated fully differentiated rod and cone photoreceptors at the dose of 0.5·10.¹⁰ vg (FIG. 6A).

In contrast, AAV-GFP injected eyes did not show positive cells for these markers. To better visualize the morphology of the differentiated photoreceptors four months after AAV-CRX treatment, a low amount of AAV-GFP (<1·10⁹) was co-injected to obtain a presence of sparse GFP-positive cells easier to image. Higher magnification of GFP-positive photoreceptors on flat-mount retina transduced with AAV-CRX demonstrated the presence of well-formed outer segments, as well as synaptic terminus (FIG. 6B).

### Example 5

Partial functional recovery in Crx^{Rip/+} retina following AAV-CRX injection.

In order to assess the functionality of the newly differentiated rod and cone photoreceptors following AAV-CRX treatment in Crx^{Rip/+}, scotopic and photopic ERG recording were performed. a statistically significant increase of the scotopic B-wave amplitude at the maximum stimulus of 2.2 Log cd sec/m² (FIG. 7A) was recorded. For the photopic response, it was overall slightly improved but statistically significant only at the stimulus of 1.6 Log cd sec/m² compared to AAV-GFP treated animals (FIG. 7B). To estimate the ability of the Crx^{Rip/+} newly differentiated photoreceptors to transduce a visual signal to the brain area dedicated to vision, a behavior named “Light/Dark choice” was used. WT mice spent a vast majority of their time in the dark side of the box whereas untreated Crx^{Rip/+} mice will go in both side indifferently due to their complete blindness. In contrast, AAV-CRX treated Crx^{Rip/+} mice spent more time in the dark side by crossing significantly less the line leading to the bright-side of the box (FIG. 7C). Altogether, these results demonstrated the ability of the AAV-CRX treatment to trigger Crx^{Rip/+} photoreceptor differentiation as well as a partial functional recovery, especially by the ability of the AAV-CRX treated mice to detect light.

### Example 6

AAV-CRX protects rd10 photoreceptors from cell death.

A potential interest of overexpressing CRX^{WT} is to stimulate the expression of its target genes. Among them, many encodes enzymes associated with IRD due to hypomorphic mutations leading to lower expression or to a low activity such as certain subtype of PDE6B mutations. Enhancing CRX target gene expression by overexpressing CRX^{WT} will allow to reach a certain threshold of expression and/or activity allowing proper phototransduction and long-term PR maintenance. This was done using the rd10 mice carrying a hypomorphic recessive mutation in Pde6b. Of note, retinal degeneration starts around P15 in this mouse model and by P30, most of the rods are lost. A sufficient increased expression level allows reaching a certain threshold of enzymatic activity sufficient to reduce cGMP levels and therefore restore the signal transduction cascade. As such, the therapy product of the invention will have a broader spectrum of application with more patients potentially treatable.

To assess the potential therapeutic effect of AAV-CRX vector for hypomorphic mutations, subretinal injection in rd10 mice at P14 (2.5×10¹⁰ vg) was performed and the retina were harvested 1 month after. IHC analysis revealed good preservation of the ONL in the treated eyes compared to controls. In contrast to non-injected eyes where outer segments are dramatically reduced, rod and cone PRs displayed well preserved outer segments where Rhodopsin and Cone Arrestin are localized such as in healthy retina (FIG. 8 (A)). Measurement of the ONL thickness confirmed the preservation (FIG. 8 (B)). In addition, the efficacy 1 months after injection at P14 with two additional doses of AAV-CRX: 0.5·10¹⁰ vg and 1·10¹⁰ vg was tested. Using paraffin section, the transduced area was determined using an anti-CRX recognizing specifically the human CRX produced by the AAV and not the endogenous mouse Crx. Measurement of the ONL thickness demonstrated that the dose of 1·10¹⁰ vg led to a better preservation of the ONL compared to the non-transduced area (FIG. 9). These results clearly demonstrated the therapeutic value of the use of the product of the invention for certain subtypes of IRD.

However due to the kinetic of degeneration starting at P14 and the time of injection, AAV-CRX injection has to be done earlier in rd10 life to improve the efficacy assessment of the therapy by giving more time for CRX production by the AAV before the onset of the degeneration.

The correct expression of the human wild-type CRX cDNA specifically in photoreceptors was demonstrated. Preliminary subretinal injections of AAV-CRX in one-month-old Crx^{Rip/+} mice demonstrated i) the absence of toxicity at the dose of 0.5·10¹⁰ vg, ii) increased CRX expression levels, and iii) reinitiation of PR differentiation four months post-injection with a large area of Rhodopsin- and Cone Arrestin-positive cells compared to non-treated mice. Notably, the gene therapy product of the invention leads to a much higher differentiation of cones compared to rod photoreceptors. Finally, a slight recovery of the scotopic and photopic electroretinogram (ERG) response 4-months after subretinal injection was observed. Together, these very promising results clearly demonstrate the validity of the product of the invention to restore PR differentiation in adult Crx^{Rip/+} retina.

### Example 6

A second mouse model was established based on the identification of two families in France each with a CORD proband carrying the CRX^{R41W} mutation. This dominant mutation does not alter CRX nuclear localization but reduces its DNA-binding properties leading to a decreased transcriptional activity of its target genes. This mutation may lead to a dominant-negative effect or to a loss-of-function preventing CRX^{R41W} from binding to target promoters. Therefore, a transgenic line, Tg(CRX^{R41W}) has been generated using a lentiviral approach (collaboration P. Charneau and L. Vives, Institut Pasteur, Paris). This line carries the recurrent human CRX^{R41W} mutation fused with a myc tag under the control of the Crx promoter. A first group of Tg(CRX^{R41W}) founders with multiple copies of the transgene showed loss of scotopic and photopic ERG responses due to photoreceptor degeneration. Successive crossing with wild-type (WT) mice to decrease the number of copies led to a partial recovery of the ERG response demonstrating the importance of the ratio between the amount of CRX^{WT} and CRX^{R41W} in the development of the disease. These observations clearly demonstrate the therapeutic interest of increasing the amount of CRX^{WT} to compensate the negative effect of the mutated CRX. Three lines with one copy of the transgene CRX^{R41W} were established wherein a normal differentiation of the photoreceptors with a slight decrease in the ERG response was found. The expression of the transgene in photoreceptors was verified by IHC.

Mice with two copies of Tg(CRX^{R41W}) have been obtained (from the line with one copy). If the line with one copy of Tg(CRX^{R41W}) had not defects in the ERG response, mice with two copies showed reduced photopic response at 6 months of age. Therefore, this line a good model of cone-rod dystrophy to test the present therapy efficacy although the degeneration is not massive and slow. To circumvent this issue, a line expressing this mutation on the Nrl^{-/-} background in which all photoreceptors are cones was established. In this line, decreased photopic response at the higher light stimulus could be observed at 2 months (FIG. 10 (A)). Preliminary injection in 2 months old animals of AAV-CRX or AAV-GFP showed preservation of the photopic response in Nrl^{-/-};Tg(CRX^{R41W}) treated with AAV-CRX compared to AAV-GFP treated animals 2 months after injection (FIG. 10 (B)).

Phenotypic Characterization of Photoreceptor Cells Differentiated from CRX-Mutated iPSC Lines of Patients and Assessment of the Efficacy of Gene Replacement.

As iPSC can be reprogrammed from fibroblasts of patients with IRDs, it is possible to generate disease-specific retinal cell models that represent excellent models for proof-of-concept gene therapy studies. iPSC-derived photoreceptor models were generated from three patients carrying CRX mutations causing CORD (p.Arg41Trp); LCA (p.Pro232Argfs*139) and RP (p.Asp65His). iPSC for the CORD and LCA patients were generated and confirmed their pluripotency and genetic stability. Furthermore, iPSC from a control and the CORD individual were differentiated into retinal organoids containing photoreceptors. Using qPCR analysis at different times of differentiation, similar expression pattern of early photoreceptor markers (SIX3, VXVX2, RAX, OTX2, NEUROD1, NRL) up to day 160 between the two lines was observed. CRX expression appeared similar until day 160, when a decrease in the CORD line compared to the control was detected. By contrast, a decreased NR2E3 expression was detected at all time points tested in the CORD line. This is particularly interesting as NR2E3 interacts with CRX for rod development. Furthermore, a decreased expression in the more mature cone (OPN1SW, CAR, OPSN1MW) and rod (RK, RCVRN) markers in the CORD line compared to control was detected Moreover, no rhodopsin expression could be detected at either the mRNA or protein level in the CORD line. These results show a delayed or altered photoreceptor differentiation in the case of the p.Arg41Trp/R41W mutation.

Patient-specific iPSC-derived photoreceptors in retinal organoids were transduced with AAV-CRX to increase wild type CRX expression, and the differentiation profile was monitored over time and compared to non-treated and control cells.

As such, i) a genotype-phenotype correlation was obtained and hence elucidated the basis for the differential clinical profiles associated with each mutation, and ii) it was determined whether all forms can benefit from a gene replacement strategy and whether there are toxic effects linked with CRX overexpression. These studies allow to assess, in the absence of in vivo administration challenges, the true effect of exogenous CRX expression.

1-13. (canceled)

14. A method for treating a CRX-associated inherited retinal disease (IRD) in a subject in need thereof, comprising administering to the subject a recombinant adeno-associated virus (AAV) vector comprising a polynucleotide encoding the retinal transcription factor cone-rod homeobox (CRX).

15. The method according to claim 14, wherein the AAV vector is an AAV2 serotype.

16. The method according to claim 15, wherein the AAV vector is an AAV2/5 or AAV2/8 serotype.

17. The method according to claim 14, wherein the polynucleotide is under the control of a promoter that drives expression in rod and cone photoreceptors chosen among Rhodopsin (Rho), beta-phosphodiesterase (PDE), retinitis pigmentosa 1 (RP1) or the human Rhodopsin kinase 1 (GRK1) promoters.

18. The method according to claim 17, wherein the polynucleotide is under the control of the GRK1 promoter.

19. The method according to claim 14, wherein the CRX-associated IRD is chosen among a retinopathy resulting from a dominant mutation in the CRX gene or results in symptoms due to a hypomorphic mutation cured by the expression of CRX.

20. The method according to claim 19, wherein the CRX-associated IRD is chosen among retinitis pigmentosa, Leber's congenital amaurosis or cone-rod dystrophies or to hypomorphic mutations in CRX target genes such as PDE6B, NMNAT1, ARL13b, AIPL1 or ABCA4 genes.

21. The method according to claim 14, wherein the AAV vector is administered to the subject in a therapeutically effective amount.

22. The method according to claim 21, wherein the quantity of the vector is between 10⁸ and 10¹² vg/eye.

23. The method according to claim 22, wherein the quantity of the vector is between 1×10⁹ and 1×10¹² vg/eye.

24. The method according to claim 14, wherein the vector is administered before disease onset.

25. The method according to claim 14, wherein the vector is administered after initiation of photoreceptor degeneration.

26. The method according to claim 14, wherein the vector is administered as long as there are functional cone and/or rod photoreceptors.

27. The method according to claim 14, wherein the vector is included in a composition.

28. The method according to claim 27, wherein the vector is included in a pharmaceutical composition.
